(19) United States
(12) Patent Application Publication
(10) Pub. No.: US 2007/0042046 A1
(43) Pub. Date: Feb. 22, 2007

(54) Methods for the treatment of cancer

(76) Inventors: Roghieh Saffie, Malvern Worcestershire (GB); Leigh Trevor Canham, Malvern Worcestershire (GB); Jonathan Charles Dormer, Malvern Worcestershire (GB); Pierce Kah Hoe Chow, Singapore (SG); Zhang Kai, Singapore (SG); Hin Robert Ng Teck, Singapore (SG)

Correspondence Address: NIXON & VANDERHYE, PC, 901 NORTH GLEBE ROAD, 11TH FLOOR, ARLINGTON, VA, 22203, US

(21) Appl. No.: 10/576,868

(22) PCT Filed: Oct. 21, 2004
(86) PCT No.: PCT/GB04/04442

§ 371 Date: Aug. 11, 2006

(30) Foreign Application Priority Data
Oct. 21, 2003 (GB) 0324483.7
Oct. 21, 2003 (GB) 0324482.9

(51) Int. Cl. A61K 9/32; A61K 31/337; A61K 31/165

(52) U.S. Cl. 424/482; 514/449; 514/618

(57) ABSTRACT

The invention provides the use of a pharmaceutical compositions comprising a cytotoxic drug and a porous carrier material in methods for the treatment of cancer by chemo-brachytherapy, in particular intra-tumoural administration.

### FIELD OF THE INVENTION

The present invention relates to methods for the treatment of cancer by chemo-brachytherapy. In particular, the invention relates to methods for the treatment of cancer by chemo-brachytherapy using pharmaceutical compositions comprising one or more cytotoxic drugs and a porous carrier material, especially for intra-tumoural administration.

### BACKGROUND TO THE INVENTION

The principal methods of treatment of cancer in common use include surgery, chemotherapy, and radiation therapy. These therapies all have serious limitations associated with their use.

Surgical resection, for example, is limited by the ability to expose and remove the tumour, and is ineffective against micro-metastases that may have migrated from the site of the primary tumour.

The effectiveness of radio- and chemotherapy is limited by the ability to target the tumour without damaging healthy tissue. In the case of chemotherapy, for example, poor specificity of the drugs for cancer cells typically results in systemic toxicity before suitable therapeutic drug levels in the tumour can be achieved. Further, as chemotherapeutic drugs usually act on rapidly dividing cells, the cells of the intestinal lining and bone marrow can be extensively damaged during the treatment. Additionally, the effectiveness of chemotherapy is often hampered by drug resistance.

Radiation therapy can be specifically directed to the site of the tumour, but is also limited by the potential damage to non-cancerous tissue. Radiation therapy relies on the ability of imaging techniques to identify tumour sites for treatment. If the tumour is too small to be imaged, it will not be identified as a target for therapy and will be left to grow and possibly metastasise.

There has been considerable interest in recent years in the possibilities of developing controlled-release or targeted-delivery options for the treatment of cancers and in particular in the development of ‘localised’ therapeutic regimens (brachytherapy), especially for late-stage cancer patients (who have already been exposed to surgery and/or a range of chemo- and radiotherapy regimens) or for the treatment of tumours associated with vital organs, glands or vasculature where surgery may not be an option.

Treatments involving the use of implants to deliver radiation and chemotherapeutic agents have been developed. Cancers treatable in this way include liver cancer, breast cancer, kidney cancer, prostate cancer and brain cancer. Radio-brachytherapy products for the localized radiotherapy of tumours are currently commercially available. Localised chemo-brachytherapy affords the possibility of a more versatile, safer and cost effective alternative method of treatment.

The use of the semiconductor, silicon, in biological applications is known in the literature and is described, for example, in WO 97/06101. Here it is disclosed that certain forms of porous silicon, in particular mesoporous silicon, are resorbable and dissolve over a period of time when immersed in simulated body fluid solution. It has been suggested that the properties of porous silicon render it useful as a vehicle for delivering beneficial substances to a subject. Where resorbable porous silicon is associated with a beneficial substance, for example, then resorption of the porous silicon in the body may result in release of the beneficial substance, affording the possibility of controlled release of the beneficial substance disposed in the pores of the porous silicon, as a result of corrosion or dissolution of the resorbable silicon.

The potential for using silicon as a delivery vehicle for an anti-cancer agent selected from one or both of a radionuclide and a cytotoxic drug is suggested in WO 02/067998. Suitable cytotoxic drugs specifically mentioned include cyclophosphamide, doxorubicin, fluoroacil, vinblastine, GNRH and cis-platin. It is suggested that implants comprising all anti-cancer component selected from a radionuclide and/or a cytotoxic drug and a silicon component may be suitable for brachytherapy by implantation of a radiation source to provide localised treatment of a tumour. Implantation of the implant into the organ in which the tumour is envisaged. Direct intra-tumoural administration is not discussed.

As yet, no effective localised chemotherapy products based on silicon as a delivery vehicle are commercially available, however, and there remains continuing interest in developing further and improved methods for localised chemo-brachytherapy of cancer tumours in order to extend the range of treatment options available to the physician.

### SUMMARY OF THE INVENTION

The invention is based on the finding that tumour regression may be achieved effectively by localised delivery of one or more cytotoxic drugs using a porous carrier material and, moreover, that formulating the drug or combination of drugs in this manner facilitates direct intra-tumoural delivery.

By means of the invention, it is possible to achieve site-specific delivery of cytotoxic drugs such as chlorambucil or paclitaxel at higher doses and with lower side effects than conventional cancer therapies. Successful localised regression of tumours permits the physician to apply more effectively secondary or tertiary therapeutic regimens for further control of the disease, thereby providing a valuable additional tool in cancer therapy. With inoperable tumours, or with tumours where resection will cause significant morbidity and loss of quality of life, localised tumour regression can downgrade the tumour volume in order to enable surgery, or simply to extend the patient's length and quality of life. The invention affords the possibility of achieving controlled release of the drug, leading to slow treatment of the tumour over a period of time. Slow regression is advantageous as the build up of necrotic tissue is reduced.

The use of the porous carrier enables improved localization of the drug in the tumour, thereby minimizing systemic toxicity, and also providing for far higher tumour-associated drug concentrations than can be achieved through conventional systemic chemotherapy. In particular, the present inventors have found that by means of the invention, it is possible to deliver cytotoxic drugs such as chlorambucil and paclitaxel at dosage levels greater than the LD50 of the free drug without significant mortality.

Chlorambucil (chemical name 4-[bis(2-chloroethyl)amino}benzenebutanoic acid) is a cytotoxic, alkylating agent which is well known for use in cancer therapy. It is used as an antineoplasic agent to treat chronic lymphatic leukemia, malignant lymphomas, giant follicular lymphoma and Hodgkin's disease and is generally available in tablet form for oral administration.

The present inventors have found that delivery of free chlorambucil ‘locally’ into tumours has limited success in restricting tumour growth and moreover that this leads to the rapid systemic distribution of the drug, resulting in systemic toxicity. Surprisingly, however, it has been found that chlorambucil when impregnated in a porous carrier system for local delivery is highly effective in effecting tumour regression, particularly when the porous carrier system carries a high loading of chlorambucil for release, in active form, at the location of the cancer.

Paclitaxel, another well known cytotoxic anticancer agent, is commercially available in injectable form (Taxol, Registered Trademark, Bristol-Myers Squibb) but pharmaceutical compositions comprising paclitaxel impregnated in a porous carrier for local delivery have not previously been described. The present inventors have found that by formulating paclitaxel in this manner, a sustained, controlled release of paclitaxel is achieved. Particularly advantageously, it has been found that, no initial ‘burst’ in the rate of release is seen, representing a significant advantage over other controlled release formulations.

According to one aspect, the invention provides the use of a pharmaceutical composition comprising a cytotoxic drug and a porous carrier material for the preparation of a medicament for the intra-tumoural delivery of a cytotoxic drug in a method of treating a cancer by chemo-brachytherapy. Also provided is the use of a porous carrier material in the preparation of such a pharmaceutical composition and a method of treating a cancer by chem-brachytherapy comprising intra-tumoural administration of a pharmaceutical composition comprising a cytotoxic drug and a porous carrier material.

In a further aspect, the invention provides the use of a porous carrier material having a cytotoxic drug incorporated into the pores of the porous carrier for the delivery of cytotoxic drug at a dose higher than the LD50 of the corresponding free drug in a method of treating a cancer.

Also provided is a method of treating a cancer in a patient by chemo-brachytherapy comprising introducing to the site at which the cancer is located a pharmaceutical composition comprising a cytotoxic drug selected from chlorambucil and paclitaxel and a porous carrier material.

The invention further provides the use of chlorambucil in the manufacture of a medicament for the treatment of a cancer by chemo-brachytherapy.

Also provided is the use of a pharmaceutical composition comprising a cytotoxic drug selected from chlorambucil and paclitaxel and a porous carrier material in the manufacture of a medicament for the treatment of a cancer by chemo-brachytherapy.

In a further aspect, the invention provides the use of a porous carrier material for the delivery of a cytotoxic drug selected from chlorambucil and paclitaxel to a patient in need thereof in a method of treating a cancer by chemo-brachytherapy.

### DETAILED DESCRIPTION OF THE INVENTION

By ‘chemo-brachytherapy’ is meant a method of treatment of cancer by localised chemotherapy in which a cytotoxic drug is introduced in or near to the tumour itself.

A cytotoxic drug may be any compound having cytotoxic properties and includes alkylating agents such as cyclophosphamide, cytotoxic antibodies such as doxorubicin, antimetabolites such as fluoroacil, vinca alkaloids such as vinbalstine and the like conventionally known in the art as anti-cancer agents. Particularly preferred cytotoxic drugs for use according to the invention include chlorambucil and paclitaxel.

It will be appreciated that the invention is intended to extend to the use of a more than one cytotoxic drug in combination therapy. Reference herein to ‘a cytotoxic drug’ will be understood accordingly to mean one or more cytotoxic drugs unless otherwise indicated. It will also be appreciated that the cytotoxic drug loaded porous carrier material may advantageously be used in conjunction with one or more other therapeutic agents. It is to be understood that the present invention covers the use of cytotoxic drug loaded porous carrier material in combination with one or more other therapeutic agents.

As used herein, a ‘porous carrier material’ is any porous material which is capable of acting as a carrier for the cytotoxic drug. The drug may be deposited on the surface of the porous carrier material or may be bound or otherwise associated with the surface of the material but preferably the drug is incorporated into the pores of the porous carrier material.

Preferably the porous carrier material for use according to the invention is capable of being loaded with high levels of cytotoxic drug distributed with high uniformity throughout substantially the entire porous material for release, in active form, at the intended site of action. Porous carrier materials which allow for release of high levels of cytotoxic drug, in active form, at the intended site of action and in a controlled manner over a period of time are particularly preferred.

The porous carrier material may suitably comprise a semiconductor, such as doped or undoped silicon carbide, silicon nitride or germanium. Preferably the porous carrier material comprises the semiconductor silicon.

Porous silicon may be classified depending upon the nature of the porosity (the porosity is the fractional void content by volume). Microporous silicon has an average pore size of less than 2 nm, mesoporous silicon has an average pore size between 2 and 50 nm and macroporous silicon contains pores having a diameter greater than 50 nm. Certain forms of porous silicon have been found to be resorbable, as described in WO 97/06101. ‘Resorbable’ relates to material that will dissolve at normal physiological temperatures (37° C.±1° C.) in simulated body fluid over a period of time, for example of up to 8 weeks and generally at less than 2 weeks. Simulated body fluid may comprise a solution of reagent grade salts in deionised water so that the ionic concentration reflects that found in human plasma or alternatively it may comprise a simulated synovial fluid, sweat or other body fluids.

Porous silicon may comprise partially oxidized porous silicon, that is, porous silicon that has been oxidized in such a manner that part of the porous silicon remains completely unoxidised.

For use according to the present invention, the porous silicon may be microporous, mesoporous or macroporous. It will be appreciated that the nature of the porosity of the porous silicon used according to the invention will depend on the intended mode of delivery but preferably the porous silicon is mesoporous.

Preferably, the porous silicon for use according to the invention has a porosity of between 1% and 99%, preferably between 20% and 90%, especially between 40% and 80%.

The porous silicon preferably comprises resorbable porous silicon. Resorption of resorbable porous silicon in the body as a result of corrosion or dissolution will result in the release of drug associated with it, affording the possibility of controlled release of the drug disposed in the pores of the porous silicon.

For a given silicon skeleton size distribution, the higher the porosity of the porous silicon, the faster it is resorbed, Depending on the choice of pore size, pore density and total volume of pore to skeleton in the porous silicon, therefore, it is possible to produce compositions which may be resorbed faster or slower, as required.

The silicon may be pure silicon or it may be doped, for example with boron. Silicon wafers are classified depending on the level of doping with either p− or p+, with p− wafers having relatively low levels of boron doping and p+ wafers having higher levels of boron doping (with resistivities in the order of 0.005 ohm.cm⁻¹). In the present invention, the silicon used is preferably derived from p+ silicon.

The porous silicon may comprise a single sample of porous silicon (in which any part of the porous silicon is substantially integral with the remaining parts of the sample), suitably of thickness between 100 nm and 1 mm, preferably between 1 micron and 750 microns. Alternatively, and preferably, the porous silicon may be in particulate form. Preferably the composition for use according to the invention comprises a multiplicity of porous silicon microparticles having a mean particle size of between 100 nm to 10 microns, preferably 500 nm to 2 microns. The multiplicity of porous particles preferably comprises a multiplicity of particles having the same shape and more preferably also the same volume as each other. The particles are each preferably substantially symmetrical and may be substantially oval, spherical or in the form of microneedles.

Porous silicon particles for use in the invention may be prepared by a number of known techniques. For example, a single crystal wafer silicon may be porosified by anodisation, for example, using HF and an electric potential. Alternatively, microparticles derived from polycrystalline feed stock may be manufactured by a two stage process, firstly by jet-milling the particle size from a few millimeters to a uniform micron sized stock followed by stain etching through established methods.

The present inventors have found that the use of compositions wherein the drug is present at a high loading level is preferred.

The compositions for use according to the invention preferably contain at least 15% by weight of cytotoxic drug, based on the weight of the composition. Preferably the drug is present in an amount of from 15% to 85% by weight, particularly from 20% to 50% by weight, especially from 30% to 45% by weight, based on the weight of the composition.

It will be appreciated that these high loading levels by weight equate to a high percentage by volume of the pores in the porous carrier material being occupied by the drug. The percentage of the maximum loading capacity that is occupied by drug (that is, the % of the total volume of the pores in the porous carrier material occupied by the drug) for compositions for use according to the invention is typically in the range of from 30% to 100%, especially from 50% to 90%. This value can be determined by dividing the volume of drug taken up during loading (equivalent to the mass of drug taken up divided by its density) by the void volume of the porous carrier material prior to loading, multiplied by a hundred.

In the compositions for use according to the invention the drug is distributed substantially uniformly throughout the pores of the semiconductor carrier material by which is meant that the porous semiconductor is impregnated with drug in an amount of from 15% to 85%, more preferably from 20% to 50%, especially from 30% to 45% by weight (based on the weight of the material) to a pore depth from the surface of the material of at least 50 microns, preferably at least 100 microns, especially at least 150 microns.

Quantification of gross loading may conveniently be achieved by a number of known analytical methods, including gravimetric, EDX (energy-dispersive analysis by x-rays), Fourier transform infra-red (FTIR) or Raman spectroscopy of the pharmaceutical composition or by UV spectrophotometry, titrimetric analysis, HPLC or mass spectroscopy of the eluted beneficial substance in solution. Quantification of the uniformity of loading is achievable by compositional techniques that are capable of spatial resolution such as cross-sectional EDX, Auger depth profiling, micro-Raman and micro-FTIR.

A preferred pharmaceutical composition for use according to the invention comprises a drug selected from chlorambucil and paclitaxel impregnated in porous silicon, wherein the drug is present in an amount of at least 15% by weight, based on the weight of the composition. Preferably the drug is present in an amount of from 15% to 85% by weight, more preferably 20% to 50% by weight, especially from 30% to 45% by weight, based on the weight of the composition The percentage of the maximum loading capacity that is occupied by the drug (that is, the % of the total volume of the pores in the porous silicon occupied by the drug) is preferably in the range of from 30% to 100%, especially from 50% to 90%.

Compositions for use according to the method of the invention having high loading levels of cytotoxic drug, such as chlorambucil or paclitaxel, distributed with high uniformity throughout substantially the entire porous material may suitably be prepared by a method comprising the steps of:
i) bringing the cytotoxic drug into contact with the porous carrier material; and
ii) allowing the cytotoxic drug to impregnate the porous carrier material, the impregnation being performed at a temperature which is at or above the melting point of the cytotoxic drug.

This may be achieved by:
i) heating the porous carrier material to a temperature at or above the melting point of cytotoxic drug;
ii) bringing the cytotoxic drug into contact with the heated porous carrier material,; and
iii) allowing the cytotoxic drug to impregnate the porous carrier material.

Alternatively, the impregnation may be brought about by the steps of:
i) heating the cytotoxic drug to a temperature at or above its melting point;
ii) bringing the molten cytotoxic drug into contact with the porous carrier material and
iii) allowing the cytotoxic drug to impregnate the porous semiconductor.

In a yet further embodiment, both the porous carrier material and cytotoxic drug may be heated independently to a temperature at or above the melting point of the drug and then brought into contact together to allow impregnation to occur.

Preferably, the porous carrier material is pre-heated to remove physisorbed moisture within the pores. Conveniently, this may be achieved by heating to a temperature in the range of from 100° C. to 250° C. for a period of from 5 to 15 minutes. The pre-heated porous carrier material is then preferably cooled down to a temperature of from 5° C. to 15° C. above the melting point of the cytotoxic drug prior to bringing it into contact with the drug and the temperature maintained for a period of time sufficient to allow impregnation of the porous carrier material to occur. Conveniently, the drug is maintained in contact with the porous carrier material for a period of from 1 minute to 2 hours, preferably from 15 minutes to 1 hour.

In a particular embodiment of the method, the cytotoxic drug is dissolved in suitable solvent before it is brought into contact with the porous carrier material. Preferably, the impregnation is carried out at a temperature which is at or above the boiling point for the solvent concerned, for example a temperature of 90° C. is suitable when ethanol is chosen as solvent and the cytotoxic drug is chlorambucil. Pre-dissolving the drug in this way leads to advantages in processing as solutions tend to be easier to handle than powders, especially where only small quantities are involved. Any solvent suitable for the drug known in the art, or mixtures thereof, may be used. Conveniently, the solvent is an organic solvent or an aqueous organic solvent, preferably an alcohol, especially ethanol or a mixture of ethanol and water

Following the introduction of the drug into the porous carrier, the treated material may conveniently be washed with a suitable solvent for the drug in order to remove any drug remaining on the surface.

Compositions for use according to the invention may be formulated for administration in any convenient manner, suitably in admixture with one or more pharmaceutically acceptable excipients. Typically, the compositions may be administered as an implant (for example subcutaneous, intramuscular, intraperitoneal or epidermal ) for introduction in any part of the patient's body in which a cancer tumour is located or for implantation into an organ such as the liver, lung or kidney. For treatment by brachytherapy, the appropriate organ may be surgically debulked and the residual space filled with composition or the organ may be cored with an array of needles and the cores filled with the composition, for example.

Preferably, the composition may be formulated for parenteral administration to be given as an injection (for example, intravenous, intravascular, subcutaneous, intramuscular or by infusion). The composition for injection may conveniently take the form of a suspension. The present inventors have found that excipients based on long and medium chain triglyercides, or derivatives of these, such as arachis oil, sesame oil and Captex® 355 (triglycerides of caprylic/capric acid) and Cremopher EL are particularly useful in parenteral formulations, as are excipients which have surfactant properties such as polysorbates.

It will be appreciated that the porous material may be formed into an implantable implant or made into particulate form either prior to or after loading with the drug.

Preferably, the compositions for use according to the invention comprise a multiplicity of microparticles, each microparticle comprising the drug and a porous carrier material.

The composition may conveniently be introduced to the site at which the cancer is located by injecting a suspension of microparticles into an artery or vein connected to or located in the organ(s) in which the tumour is located. This provides for an even distribution of drug throughout the affected area.

In a particular embodiment, the method according to the invention may comprise direct intratumoural introduction of the composition. Compositions in the form of microparticles may be delivered using a fine bore needle and this represents a significant advantage in terms of ease of administration.

In order accurately to guide the introduction of the pharmaceutical composition according to the method of the invention, the tumour may be imaged using a number of known techniques such as computer tomography, ultrasound, positron emission tomography and magnetic resonance tumour imaging.

The method of the invention is applicable to many cancers including liver cancer, breast cancer, kidney cancer, prostate cancer and brain cancer. In particular, it is of interest in the treatment of liver cancer for which there are at present few therapeutic options.

The present invention may be illustrated by the following non-limiting examples, when read together with the accompanying figures in which:—

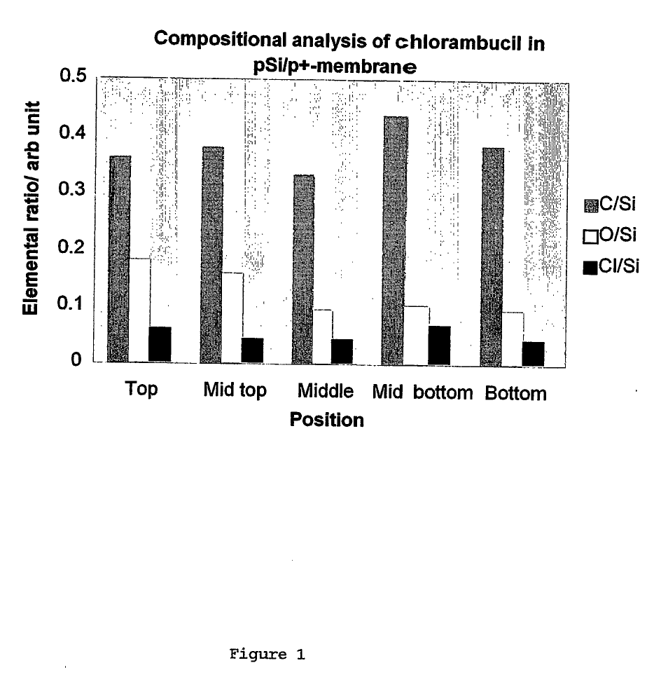

FIG. 1 shows the compositional analysis of a porous silicon p+ membrane impregnated with chlorambucil, as determined by SEM-EDX, in which with reference to the total thickness of the membrane (˜160 μm),
Top refers to EDX positioned at 1-5 μm from the polished side of membrane
Mid. Top is referred to 35-45 μm from the polished side of membrane
Middle is referred to 75-85 μm in between unpolished and polished side of membrane
Mid. Bottom is referred to 35-45 μm from the unpolished side of membrane
Bottom is referred to EDX positioned at 1-5 μm from the unpolished side of membrane

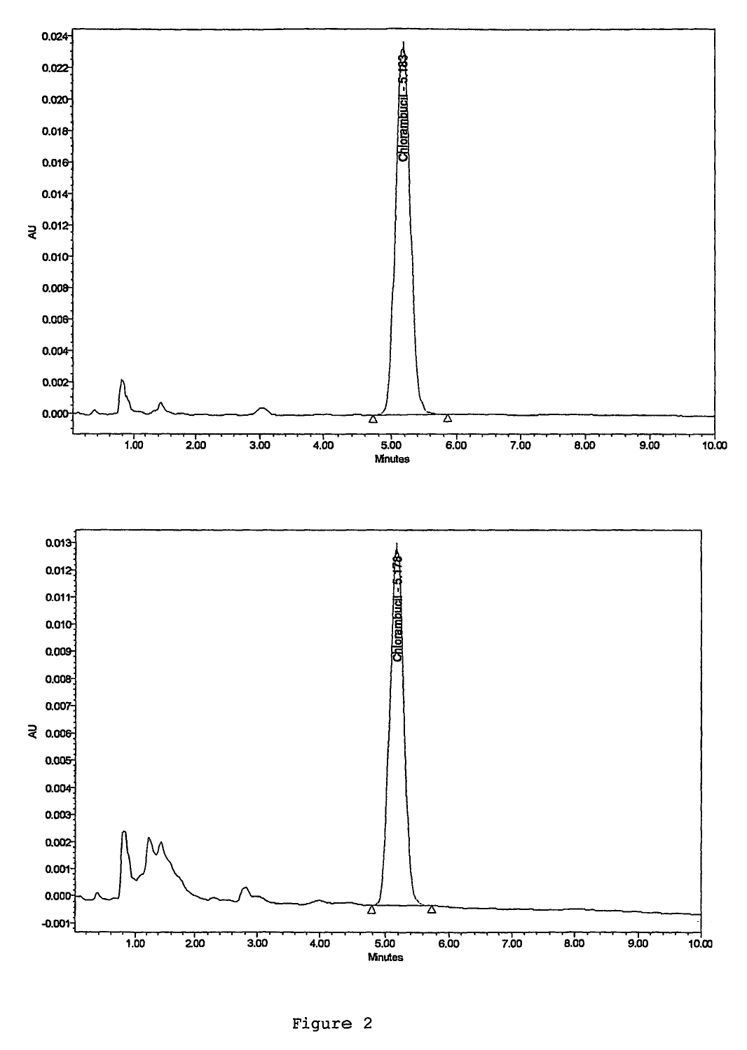

FIG. 2 shows an HPLC analysis of chloambucil, standard in ethanol (A) and after incorporation and extraction from porous silicon (B)

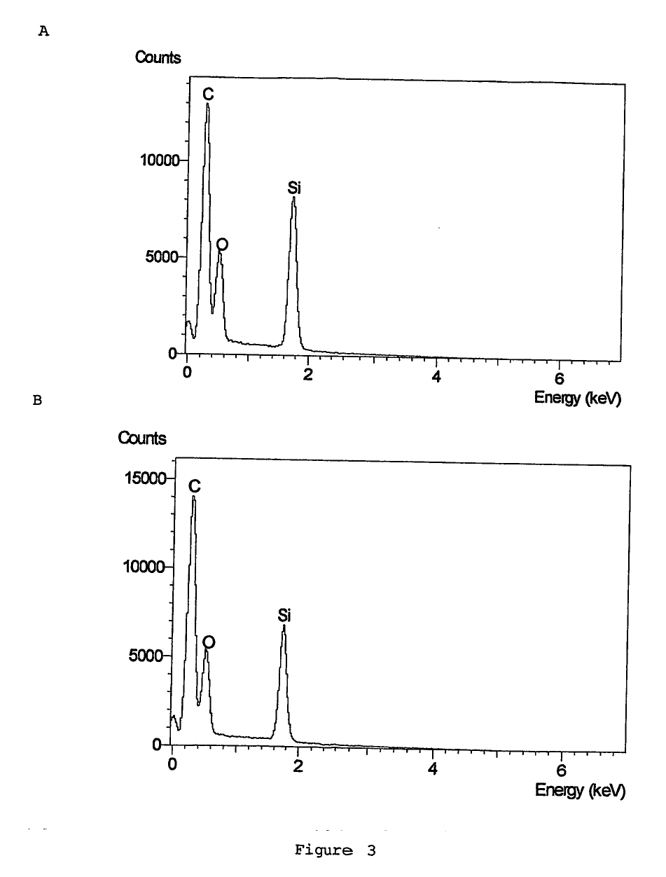

FIG. 3 shows the SEM-EDX analysis of a paclitaxel impregnated porous silicon/p+ membrane performed on the cross-section of the membrane at a depth of (A) 80 μm and (B) 145 μm from the surface of the material.

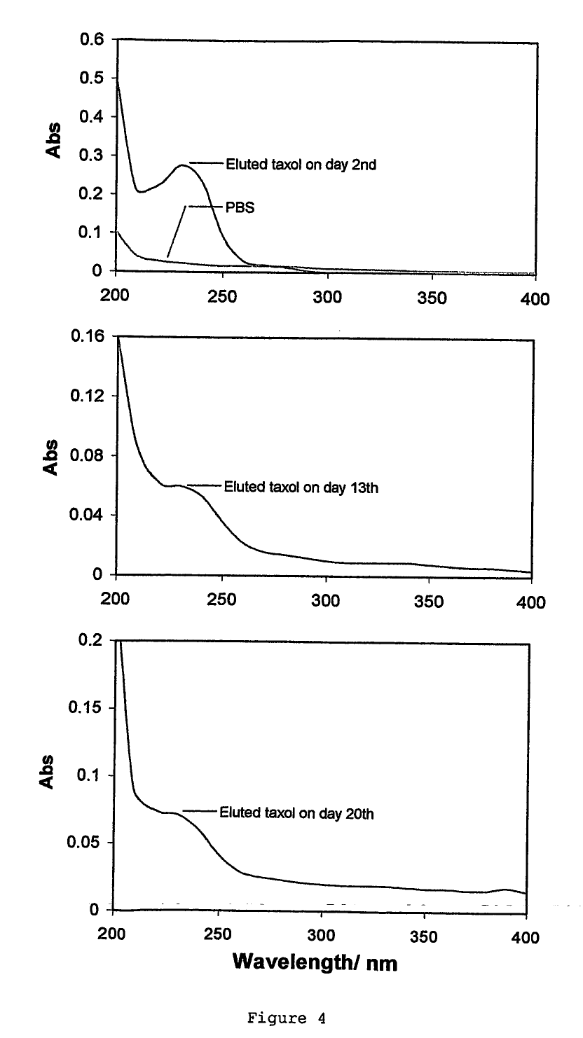

FIG. 4 shows the UV profiles obtained from PBS eluted samples of paclitaxel impregnated porous silicon membrane on days 2 (A), 13(B) and 20 (C).

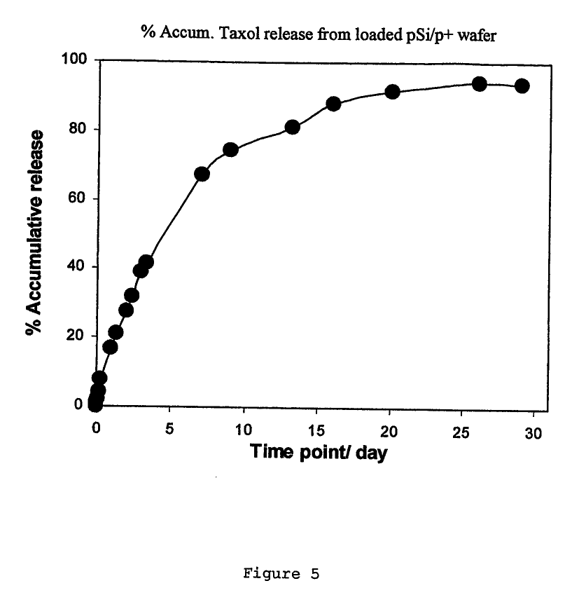

FIG. 5 shows the graph obtained for the cumulative release of paclitaxel from impregnated porous silicon membrane measured over a period of 30 days.

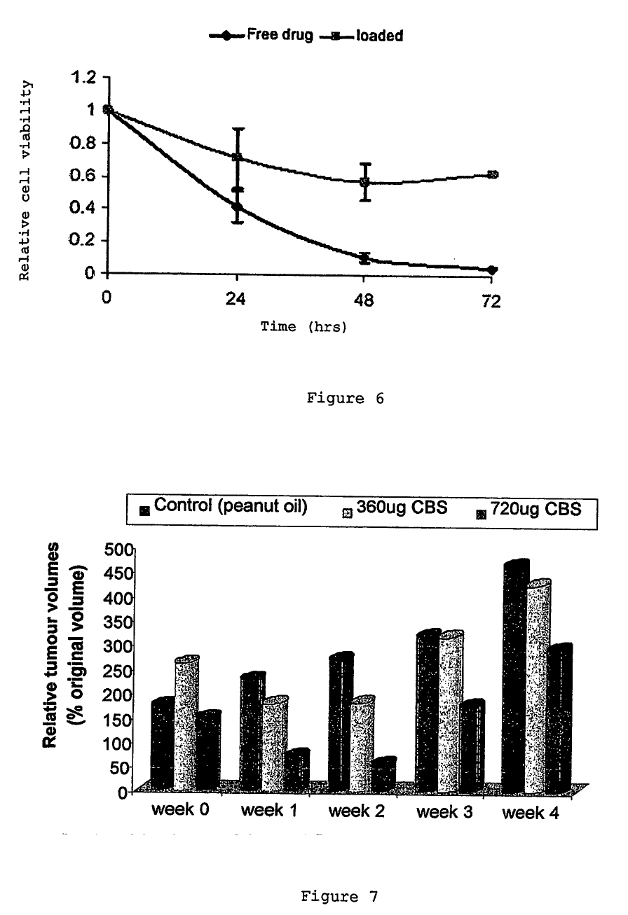

FIG. 6 shows the cytotoxity profile over time of chlorambucil (120 ug) alone or impregnated into porous silicon membrane (=p<0.05)

FIG. 7 shows the effects on tumour regression following intratumoural injection of two dosages of chlorambucil impregnated into porous silicon membrane (CBS). The control group received intratumour injection of 100 ul of peanut oil only.

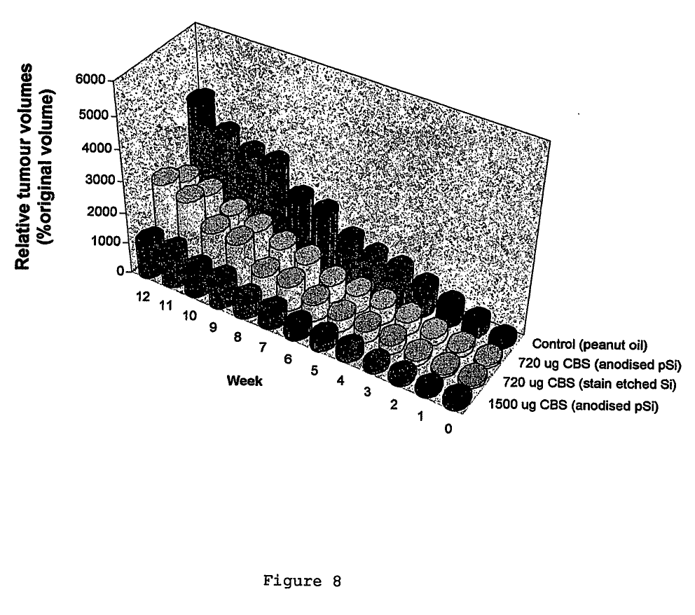

FIG. 8 shows the dose-dependent regression of human tumour in mice following intratumoural injection of chlorambucil delivered by porous silicon. (CBS). The control group received intratumour injection of 100 ul of peanut oil only.

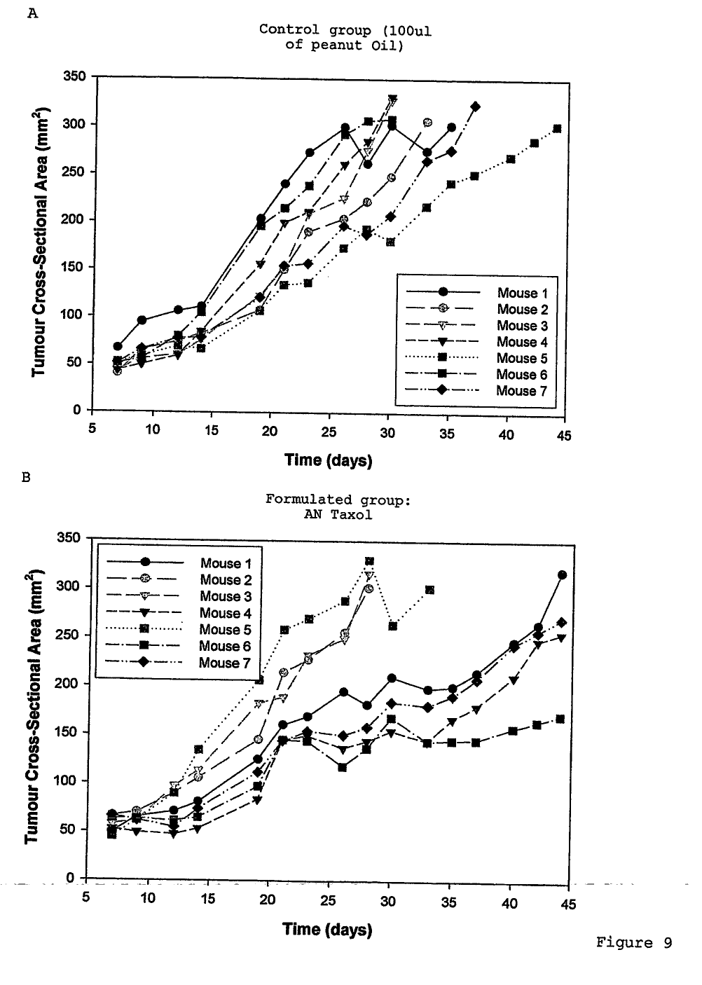

FIG. 9 shows a graph of tumour cross-sectional area over time in mice following intratumoural administration of peanut oil (A) and paclitaxel loaded porous silicon (B)

### EXAMPLE 1

Preparation of Porous Silicon Microparticles.

Porous silicon microparticles were prepared from either single crystal wafer silicon (purity 99.99999%) or from polycrystalline feed stock material (purity 99.999%). In the first case, silicon surfaces were porosified in a double-tank anodisation cell. Samples of P-type boron doped silicon wafer were anodised in a solution of HF: EtOH. The samples were rinsed with ethanol and dried by using a spin dryer. The resulting pore size distribution was monitored by high resolution SEM microscopy (JEOL 6400F) and the thickness of the porous layer was measured using cross sectional SEM.

Microparticles derived from polycrystalline feed stock were manufactured by jet-milling the particle size from a few millimeters to a uniform micron sized stock followed by stain etching using conventional methods. Porous silicon microparticles derived from stain etching were poly-Si mm spheroids jet milled to 4 micron diameter and then reacted in a staining solutions containing water/HF/Nitric acid for typically 3-30 minutes. This generated powder with a typical surface area of 50-100 m2/g.

### EXAMPLE 2

(i) Combination of Porous Silicon Microparticles with Chlorambucil.

i) Stain Etched Porous Silicon

Chlorambucil is dissolved in ethanol (50 ml, added to an accurately weighed amount of stained etched porous silicon in a round bottomed flask and the mixture placed on a rotary evaporator and rotated without applying the vacuum for typically 30minutes to ensure that the chlorambucil solution has infused into all of the pores in the material.

After this time a gentle vacuum is applied to the rotating round bottom flask to gently distil off the ethanol, leaving chlorambucil incorporated into the porous silicon. During the gentle vacuum, the round bottom flask is placed in water bath having temperature around 50C and inside a flask the temperature was above boiling point of solvent (around 67C for ethanol).

2. Anodised Porous Silicon

A porous silicon flake (porosity 63%) prepared according to the anodisation method of Example 1 was pre-heated to 100-250° C. for 5-15 minutes to remove the majority of physisorbed moisture within the pores. The flake was then cooled to a temperature of 80° C. (10 ° C. above the melting point of chlorambucil) for 5 minutes. The flake was removed and washed briefly in ethanol. Chlorambucil was then immediately placed in powder form on the uppermost face of the flake and the temperature was maintained for 25 minutes. The flake was removed and washed in ethanol

After the drug loading and washing its weight increased to 72.92 mg equivalent to a w/w/loading of 39.19%. Thus the drug occupied 88.40/drug density of the pore volume available.

$$Weight of unloaded flake = 44.34 mg$$

$$Density = {d}_{chl} g {cm}^{- 3}$$

$$\begin{matrix}Void volume = \{ 44.34 / 479.60 mg \} \times 814.75 mg / \{ density of silicon \} \\ = 0.03233 {cm}^{3}\end{matrix}$$

$$\begin{matrix}Maximum loading capacity = 0.03233 {cm}^{3} \times {d}_{chl} g {cm}^{- 3} \\ = 32.33 {d}_{chl} mg\end{matrix}$$

Loading & Washing

$${w}_{1} = 71.66 mg$$

$${w}_{2} = 73.41 mg$$

$${w}_{3} = 72.92 mg equivalent to 39.19 \% w / w$$

$$\begin{matrix}\% loading capacity ( w / v ) = \{ 72.92 - 44.34 \} / \{ 32.33 {d}_{chl} \} \times 100 \\ = 88.40 / {d}_{chl} \%\end{matrix}$$

Compositional analysis of the impregnated membrane, using the SEM-EDX method (FIG. 1), revealed that the loading of chlorambucil is fairly homogenous through the membrane, with reference to C/Si and Cl/Si ratios. High chlorambucil is detected, as evidenced with C/Si˜0.35 on average.

Following incorporation into the membrane, chlorambucil was extracted and subjected to HPLC analysis. A known amount of the incorporated Chlorambucil/porous silicon (typically 10-15mg, dependent on the level of incorporation) was placed into a volumetric flask and made up to a volume with 100 ml of ethanol, shaken and sonicated for 30 minutes at 30° C. From this stock solution 25 ml were transferred by pipette to a 100 ml volumetric flask to give the sample solution. Prior to analysis by HPLC the sample was filter through a 0.45 micron filter.

The chromatographic conditions used for this analysis were:

| Parameter | Condition |
|---|---|
| Mobile Phase | Methanol:Water:Acetonitrile:Glacial |
| | Acetic Acid 58:39:2.5:0.5 |
| Column | Waters Xterra RP18 3.5 μm 4.6 × 100 mm |
| Column Temperature | 35° C. |
| Sample chamber temperature | 15° C. |
| Injection Volume | 20 μl |
| UV absorbance | 257 nm |
| Needle Wash | Methanol:Water 50:50 |
| Run Time | 10 minutes |
| Retention time Chlorambucil | Approximately 5.0 minutes |
| Calibration Type | Using external Chlorambucil standards to |
| | create linearity curve |

The results (FIG. 2) demonstrated that the compound could be incorporated and released from the porous silicon material without significant degradation. An unchanged position of the single dominant peak labelled chlorambucil is the main indicator that insignificant degradation has occurred as a result of loading into and subsequent release from porous silicon. Appreciable degradation would have resulted in additional intense peaks around the parent peak at 5.2 minutes “retention time” in the spectrum. The large number of small peaks between 0 and 3.2 minutes retention time are unrelated to chlorambucil.

(ii) Combination of Porous Silicon Microparticles with Paclitaxel

Following the method described above, impregnation of the porous silicon membrane by a solution of the drug in ethanol using the rotary evaporation method gave a drug loading equivalent to 16.71% w/w.

SEM-EDX analysis of the paclitaxel impregnated porous silicon/p+ wafer (166 μm thick) confirms that the paclitaxel is substantially uniformly distributed throughout the membrane, as can be seen from the EDX spectra performed on the cross-section of the membrane at a depth of (A) 80 μm and (B) 145 μm from the surface of the material presented in FIG. 3.

HPLC analysis confirms that the compound is incorporated into and release from the porous silicon material without significant degradation.

The dissolution of the paclitaxel impregnated porous silicon membrane (weight of sample equivalent to 2 mg porous silicon) was studied in a total media replacement dissolution apparatus with PBS (phosphate buffer solution, pH 7.4) as dissolution medium at ambient temperature using UV spectroscopy (230 nm). At each time point (each day) the impregnated membrane was transferred from one 4 ml PBS containing bijou into a fresh 4 ml PBS containing bijou.

FIG. 4 shows the UV profiles obtained from eluted samples on days 2, 13 and 20 (A-C respectively). From these results it can be seen that the absorption profile of the eluted drug does not alter over the period of 20 days, suggesting that the drug eluted from the loaded sample is not substantially degraded over this time.

FIG. 5 shows the graph obtained for the cumulative release of paclitaxel from the impregnated membrane measured over a period of 30 days. From this it can clearly be seen that sustained, controlled release of paclitaxel is achieved, the release rate being approximately constant for the first 7 days and slowing thereafter until after 30 days, 95% of the drug has been recovered. Particularly notably, no initial ‘burst’ in the rate of release is seen, this represents a significant advantage over other controlled release formulations.

### EXAMPLE 3

In vitro Studies on Cell Viability of Chlorambucil Loaded Porous Silicon Microparticles

(i) Studies were carried out using a Neutral Red Assay.

Cells were cultured according to the following protocol:

1. Cells are seeded in 1 ml media/well in 24 well plates at the appropriate density (pre-determined e.g. for Hela's=40,000/well) so that cell confluency is 60-70% at the start of the experiment.

2. Plates are then left overnight at 37° C., 5% CO₂ to adhere.

3. Trans-well inserts are added to each well of the 24 well plates prior to preparation of the formulations.

4. Samples of porous silicon impregnated with chlorambucil were weighed out and suspended in Mc Coy's 5a medium, supplemented with 10% fetal calf serum (FCS), 2 mM Glutamine. Controls were set up with medium containing no chlorambucil and with medium containing free chlorambucil, solubilised in ethanol. 100 μL of each formulation was added to each appropriate trans-well insert in triplicate and returned to the incubator for 24 hrs prior to analysis of viability.

The Neutral Red assay used is described by Triglia, D., et al, In Vitro Cell. Dev. Biol. 27A, 239 (1991) and Fautz, R., et al., Mutat. Res. 253, 173-179 (1991).The following procedure is followed:

1. A 4 mg/ml neutral red stock in water is used to prepare a 50 μg/ml working solution in pre-warmed culture media, allowing 0.5 ml per well for treatment.

2. Inserts & culture media are removed from each well, cells gently washed with PBS and neutral red solution added followed by incubation at 37° C. for 45 mins.

3. Dye is then removed, cells re-washed with PBS×2 to remove unincorporated dye & 0.5 ml destain solution (50% water, 49% Ethanol, 1% Glacial acetic acid) added to each well to solublise all incorporated dye.

4. 90 μL of each sample is then aliquoted in triplicate into a 96 well plate (including destain control) & absorbance of the plate at read at 540 nm

5. Neutral Red uptake of the treated wells is expressed relative to the untreated ones to give relative cell viability.

The results (FIG. 6) show that porous silicon impregnated with chlorambucil showed the same pattern of cytotoxicity as the free drug, but with a clear delay in release. This data was collected using endometrial (ISH) cells and large porous silicon flakes prepared by anodisation and loaded with chlorambucil (120 ug) by the melting technique. Accompanying HPLC data (not shown) on chlorambucil incorporation and release confirmed that much longer culture conditions were required to completely release the drug payload corresponding to the free drug. This is why more than 60% of the cells are still viable after 72 hrs for the loaded drug, rather than loaded chlorambucil being less cytotoxic due to degradation. Similar delayed cytotoxicity profiles were also obtained with fibroblast (3T3s) and intestinal cells at loading levels up to 240 ug/ml of chlorambucil in culture media.

(ii) The cytotoxic effects of chlorambucil loaded porous silicon on the HL-60 cell line, derived from a single patient with acute promyelocytic leukemia, was assessed. This cell line provides a unique in vitro model system for studying the cellular and molecular events involved in the proliferation and differentiation leukaemic cells of the granulocyte/monocyte/macrophage lineage.

Cells with formulated drug were collected at 24 hours after incubation, placed into sterile plastic tubes and centrifuged. The supernatants were aspirated out and the cell pellets were washed gently with copious amount of PBS and centrifuged to remove any excipient. The cell pellet was dislodged gently and incubated with 300 μl of Live/dead cell assay reagent and incubated for 45 minutes on a rocker with gentle shaking (20 shakes per minute). At the end of incubation the cells were spun down, washed twice with PBS, reconstituted with 1 ml of PBS and transferred into a plate to capture the images of Live/Dead cells using fluorescent microscope.

The Live/Dead cell assay was performed on HeLa cell cultures grown on collagen coated plated and treated with various forms of porous silicon (AN⁺/SE, loaded/unloaded, pre-formulated/formulated). The results show that there were only 1.2% of dead cells in untreated group. When HeLa cells were treated with peanut oil formulated unloaded AN⁺/SE porous silicon, there was no increase in cytotoxic effect on these cells (0.8 and 2.8 % respectively and it is statistically not significant when compared to untreated). However, when these cells treated with formulated AN⁺/SE porous silicon loaded with chlorambucil (90 μg/ml), the dead cell percentage increased to 16/10 respectively, indicating some amount of cytotoxicity and it is statistically significant when compared to untreated and unloaded group. Unformulated AN⁺/SE porous silicon loaded with chlorambucil (90 μ/ml) had considerable cytotoxic effect on HeLa cells and the cytotoxicity was increased to 38.4/18.4 percent respectively on these cells. The increase was statistically significant. When HeLa cells were treated with free drug (90 μ/ml) dissolved either in culture medium or peanut oil had very severe cytotoxicity effect and the percentage of cytotoxicity observed was 95% and above.

The live and Dead cell assay performed after treating HeLa cells with various forms of porous silicon and the results interpreted. The procedure was performed on adherent cells after the floating cells were washed off. It is evident from the above results that the dead cells in free drug alone experiments are stuck to the bottom of the plate and the number is almost equivalent to the number of cells observed in any other experimental condition. Therefore, percentage of live and dead cells was calculated from the adherent cells stuck to the bottom of the wells. Each sample was run in triplicate wells and one microscopic field image from each well was captured and the live and dead cells were counted to get an average % of cytotoxicity. The results suggest from formulated unloaded porous silicon experiments that unloaded porous silicon by itself does not cause any toxicity. The formulated and loaded AN⁺ and SE samples could produce only 10-16% of cytotoxicity when compared to free drug alone, though the amount of chlorambucil present in each experiment remains the same. The results indicate that the drug is being released very slowly from formulated and loaded porous silicon. However the cytotoxic effects of unformulated and loaded samples was 19-36% suggesting that the drug may be released slowly though the amount of chlorambucil present is the same as in free drug experiments. In all the above experiments AN⁺ porous silicon seem to be releasing optimum drug than SE porous silicon and more effective in producing cytotoxicity in 24 hours treatment experiments.

The unloaded formulated SE porous silicon did not produce any toxicity, the percentage of dead cells were not statistically significant when compared to untreated group. However, both AN⁺ and SE formulated porous silicon loaded with chlorambucil produced considerable toxicity (23 and 48 percent of dead cells respectively).

### EXAMPLE 4

In vivo Studies

The effects of intra-tumoural administered chlorambucil loaded porous silicon and paclitaxel loaded porous silicon formulations, prepared as described above, on tumour growth in mice were studied. In both cases, effective tumour regression was observed without significant mortality, even when the drug is administered at a dosage level greater than the LD50 of the corresponding free drug.

i) Chlorambucil

Studies on animal models were carried out in the animal laboratory of the Dept of Experimental Surgery of the Singapore General Hospital with the approval by the Ethical Committee of the Singapore General Hospital.

The following protocol was adopted:
1. Culture of human cells and implantation of the tumour cells as solid tumours in nude mice: The carcinoma cell-line was cultured in RPMI and Ham's F-12 media respectively with 10% fetal calf serum in a CO₂ incubator and subcultured every two to three days until growing to a number enough for implantation in animals. The cells were then implanted as solid tumors in nude mice]. The cells were collected in HBSS (Hanks balanced salt solution). One hundred microliters of cell suspension in HBSS (5×10⁶ cells) was injected subcutaneously into right gluteal region of nude mice (female, 6 to 9 week old nude mice with average body weight of 25 g, housed in pathogen-free conditions) using a 25-gauge needle.
2. Animals groups: Animals were randomly grouped into control 1 (injected with peanut oil), control 2 (injected with porous silicon without drug), free chlorambucil (drug injected without porous silicon directly into tumour or injected via intraperitoneal route) and chlorambucil impregnated porous silicon groups. Each group included 16 to 20 animals.
3. Injection of chlorambucil impregnated porous silicon in transplanted tumours: On day 14 after implantation of tumours (diameter of the tumour about lcm), the chlorambucil impregnated formulation was applied to the centre of the tumours.
4. Tumour volumetrics: The sizes of implanted tumours in nude mice was estimated every 3 days. The largest and smallest diameters were measured by a vernier caliper and tumour volumes estimated according the formula: V=½ ab², where a and b are largest and smallest tumour diameters respectively, and V is the tumour volume in cm³.

The effects of intra-tumoural administered chlorambucil loaded porous silicon on the growth of the tumour was studied and compared with control and systemic therapy groups. The body weight of the animals was estimated by subtracting the tumour volume (cm3) from the total body weight (g) every 3 days. The survival time of each experimental animal was recorded.

As can be seen from the results of the relative tumour volumes over time presented graphically in FIG. 7, chlorambucil loaded into porous silicon (CBS) was very effective in causing tumour regression depending on the dosage used. These results are from anodisation derived porous silicon flake loaded with chlorambucil (360 ug or 720 ug) by the melting solvent method and subsequently subjected to particle size reduction to about 20 microns by grinding in a pestle and mortar for 1 hour

In another series of experiments using the same tumour model, higher dosage of chlorambucil (1500 ug), delivered by porous silicon and injected directly into the tumour, significantly decreased the size of the tumour when compared with the animals that had received no treatment (control group). Flank tumour growth was delayed by 12 weeks when compared with the animals that had received no treatment (FIG. 8). In contrast, intratumour delivery of chlorambucil using the same dosage (2×LD50) resulted in a 90% animal mortality rate. An increase of the drug dosage from 720 ug (LD50) to 1.5 mg (2×LD50) led to 20% mortality in the CBS groups suggesting the possibility of prolonged tumour exposure to the drug while minimizing the drug's dose-limiting, systemic side effects. In contrast, the administration of similar amounts of Chlorambucil without porous silicon resulted in a mortality rate of 50% (720 ug) to 90% (1500 ug).The results of these studies showed a significantly prolonged survival rate for the group treated according to the method of the invention. Moreover, treatment by locally administered chlorambucil loaded porous silicon produced long-term survivors

Paclitaxel

The effect of paclitaxel loaded porous silicon on the subcutaneous growth of the human breast carcinoma MCF7 in nude mice was investigated and compared to a control group of mice injected with peanut oil excipient only.

Studies were performed at the University of Nottingham. The following protocol was adopted:

Implantation method: The tumour line was maintained in serial passage in nude mice. For the therapy study, donor mice were sacrificed and the tumours excised. The tumour was finely minced, and 3 mm³ sections implanted subcutaneously into the flank of MF1 nude mice under suitable anaesthesia. Animals were examined regularly for the appearance of tumours. When measurable tumours had established, the mice were assigned to the treatment groups by size to give a representative distribution of sizes within each group. Tumour size was evaluated three times weekly.

The treatment group received 100 ul of formulated compound (paclitaxel loaded into porous silicon) injected directly to tumour. The control group received 100 ul of peanut oil only.

From the results of the determination of tumour cross-sectional area over time presented graphically in FIG. 9, it can be seen that retardation of tumour growth is observed in mice treated with paclitaxel loaded porous silicon (B) when compared with the control group (A).

1. Use of a pharmaceutical composition comprising a cytotoxic drug and a porous carrier material in the preparation of a medicament for the intra-tumoural delivery of a cytotoxic drug in a method of treating a cancer by chemo-brachytherapy.

2. Use according to claim 1 wherein the porous carrier material is doped or undoped silicon, germanium, silicon carbide or silicon nitride.

3. Use according to claim 1 wherein the porous carrier material is silicon.

4. Use according to claim 3 wherein the silicon is resorbable

5. Use according to claim 4 where the resorbable silicon is mesoporous.

6. Use according to claim 1 wherein a cytotoxic drug is incorporated into the pores of the porous carrier material.

7. Use according to claim 1 wherein the cytotoxic drug is present in an amount of from 15% to 85% by weight, based on the weight of the composition.

8. Use according to claim 1 wherein the cytotoxic drug is selected from chlorambucil and paclitaxel.

9. Use according to claim 1 wherein the pharmaceutical composition comprises a multiplicity of microparticles.

10. Use of a porous carrier material in the preparation of a medicament for intra-tumoural delivery of a cytotoxic agent.

11. A method of treating a cancer by chemo-brachytherapy comprising intra-tumoural administration of a pharmaceutical composition comprising a cytotoxic drug and a porous carrier material.

12. A method according to claim 11 wherein the pharmaceutical composition is as defined above.

13. Use of a porous carrier material having a cytotoxic drug incorporated into the pores thereof to delivery a cytotoxic drug at a dose higher than the LD50 of the corresponding free drug in a method of treating a cancer.

14. Use according to claim 13 wherein the cytotoxic drug is selected from chlorambucil and paclitaxel.

15. Use of chlorambucil in the manufacture of a medicament for the treatment of a cancer by chemo-brachytherapy.

16. Use of a pharmaceutical composition comprising a porous carrier material and a cytotoxic drug selected from chlorambucil and paclitaxel in the manufacture of a medicament for the treatment of a cancer by chemo-brachytherapy.

17. Use of a porous carrier material to deliver a cytotoxic drug selected from chlorambucil and paclitaxel in a method of treating a cancer by chemo-brachytherapy.

18. A method for treating a cancer by chemo-brachytherapy comprising introducing to the site at which the cancer is located a pharmaceutical composition comprising a porous carrier material and a cytotoxic agent selected from chlorambucil and paclitaxel.

19. A method according to claim 18 wherein the porous carrier material is silicon.

20. A method according to claim 18 wherein the pharmaceutical composition is introduced to the site at which the cancer is located by injecting a suspension of microparticles into an artery or vein connected to or located in the organ in which the cancer tumour is located.

21. A method according to claim 18 wherein the pharmaceutical composition is introduced to the site at which the cancer is located by injecting a suspension of microparticles into the cancer tumour.
